(19) United States
(12) Patent Application Publication
(10) Pub. No.: US 2020/0239842 A1
(43) Pub. Date: Jul. 30, 2020

(54) Preparation and Expansion Methods for Human Pluripotent Stem Cell-Derived Human Retinal Pigment Epithelial Cells

(71) Applicant: ZHONGSHAN OPHTHALMIC CENTER, SUN YAT-SEN UNIVERSITY, Guangzhou (CN)

(72) Inventors: Xiufeng ZHONG, Guangzhou (CN); Jian GE, Guangzhou (CN); Shengxu LIU, Guangzhou (CN); Fuhua PENG, Guangzhou (CN)

(73) Assignee: ZHONGSHAN OPHTHALMIC CENTER, SUN YAT-SEN UNIVERSITY, Guangzhou (CN)

(21) Appl. No.: 16/308,830

(22) PCT Filed: Apr. 28, 2018
(86) PCT No.: PCT/CN2018/085026

§ 371 Date: Dec. 11, 2018

(30) Foreign Application Priority Data
Jun. 13, 2017 (CN) 201710441265.9

(51) Int. Cl. C12N 5/079

(52) U.S. Cl. CPC C12N 5/0621; C12N 2513/00; C12N 2509/10; C12N 2509/00; C12N 2502/45; C12N 2500/30; C12N 2500/32; C12N 2500/00; C12N 2533/90

(57) ABSTRACT

Preparation and expansion methods for human pluripotent stem cell-derived human retinal pigment epithelial cells are provided. The preparation method includes the following steps: collecting 3D-PRE spheroids derived from human pluripotent stem cells, performing mechanical separation to remove non-RPE cells and clusters which are non-pigmented and retain a pigmented RPE cell sheet, enzymatically digesting the pigmented RPE cell sheet to obtain an RPE single cell suspension, and thereby the human pluripotent stem cell-derived human retinal pigment epithelial cells are obtained. The expansion method includes centrifuging the RPE single cell suspension and removing a supernatant, resuspending in an RPE cell medium and seeding into a cell culture container precoated with extracellular matrix to allow primary culture, and subculturing the cells after cells spread out.

### CROSS REFERENCE TO THE RELATED APPLICATIONS

This application is the national phase entry of International Application No. PCT/CN2018/085026, filed on Apr. 28, 2018, which is based upon and claims priority to Chinese Patent Application No. CN201710441265.9, filed on Jun. 13, 2017, the entire contents of which are incorporated herein by reference.

### TECHNICAL FIELD

The present invention relates to the technical field of stem cells, and particularly relates to preparation and expansion methods for human pluripotent stem cell-derived human retinal pigment epithelial cells.

### BACKGROUND

The retinal pigment epithelium (RPE) is located outside the retinal neuroepithelium, providing nutrients to the latter and participating in phototransduction reactions. Degeneration, death and dysfunction of PRE are prominent causes of retinal degenerative eye diseases. Retinal pigment epithelial cell transplantation is one of the most promising methods for restoration of visual function, but the lack of RPE cells has limited the development of this treatment. Prior to the rise of stem cell technology, the source of RPE cells was limited to the isolation from eyeballs of early aborted embryos or voluntary donor. Recent studies have shown that human pluripotent stem cells (hPSCs), including human embryonic stem cells (hESCs) and human induced pluripotent stem cells (hiPSCs), are capable of differentiating towards retinal pigment epithelium cells. RPE cells derived from hPSCs are the most promising seed cells for the treatment of retinal degenerative diseases. There are mainly two methods for induced differentiation of pluripotent stem cells into RPE cells, the traditional two-dimensional adherent culture protocol and the three-dimensional retinal induction protocol. However, RPE cells produced by these methods are always mixed with other non-RPE cells produced during differentiation of hPSCs. Therefore, how to isolate and purify the RPE cells in order to obtain RPE seed cells for related research or treatment is a technical problem that needs to be solved.

### SUMMARY

One object of the present invention is to overcome the deficiencies in the prior art and provide simple and widely applicable preparation and expansion methods for human pluripotent stem cell-derived human retinal pigment epithelial cells.

The preparation method for human pluripotent stem cell-derived human retinal pigment epithelial cells of the present invention, comprises the following steps: collecting 3D-PRE spheroids derived from human pluripotent stem cells, performing mechanical separation to remove non-RPE cells and clusters which are non-pigmented and retain a pigmented RPE cell sheet, enzymatically digesting the pigmented RPE cell sheet to obtain an RPE single cell suspension, and thereby the human pluripotent stem cell-derived human retinal pigment epithelial cells are obtained.

The pluripotent stem cell-derived RPE cells prepared by the method of the invention show characteristics closely similar to those of the human fetal RPE cells, that they have typical RPE cell morphology, express specific molecular markers PAX6, OTX2, ZO-1 and RPE65, and exhibit normal physiological functions such as polarized secretion of cytokine PEDF.

The human pluripotent stem cells are preferably human embryonic stem cells or human induced pluripotent stem cells. These two types of cells can be cultured using known methods.

The 3D-PRE spheroids are prepared by: inducing directed differentiation of the human pluripotent stem cells into retinal cells including RPE cells, collecting adherent cells including the RPE cells by scraping, and culturing them in suspension to obtain the 3D-RPE spheroids. The 3D-RPE spheroids may be free-floating, or adherent to one side of a neural retinal cup, or adherent to one side of another cell cluster.

The 3D-RPE spheroids are preferably selected from 3D-RPE spheroids prepared by induced differentiation of human pluripotent stem cells for more than 40 days, wherein the day of initiation of the differentiation (when the expansion medium of the hPSC is replaced with a differentiation medium, or when embryoid bodies are prepared) is regarded as day 0 of the differentiation.

The step of performing mechanical separation to remove non-RPE cells and clusters which are non-pigmented and retain a pigmented RPE cell sheet, is specifically as follows: transferring all the 3D-RPE spheroids into a cell culture container, digesting in a 37° C. water bath for 8-15 minutes using a digestive reagent, washing with 1×PBS for several times after the digestive reagent was aspirated, separating the pigmented RPE cell sheet from the non-RPE cells and clusters which are non-pigmented using a tungsten needle, and retaining the pigmented RPE cell sheet.

The digestive reagent is preferably a Dispase II solution having a mass fraction of 1-2%.

The step of enzymatically digesting the pigmented RPE cell sheet to obtain an RPE single cell suspension, is specifically as follows: transferring the pigmented RPE cell sheet to a TrypLE Express solution in a 37° C. water bath to allow digestion for 7-10 minutes, centrifuging to remove the digestive reagent, resuspending in an RPE cell medium, and filtering the cells through a 70-100 μm strainer to obtain the RPE single cell suspension.

The present invention further provides an expansion method for human pluripotent stem cell-derived human retinal pigment epithelial cells, comprising the following step: centrifuging the above-mentioned RPE single cell suspension and removing a supernatant; resuspending in an RPE cell medium and seeding into a cell culture container precoated with extracellular matrix to allow primary culture; after cells spread out, subculturing the cells to obtain the human pluripotent stem cell-derived human retinal pigment epithelial cells.

Preferably, the step of seeding for primary culture is performed at a cell density greater than or equal to 5×10⁴ cells/cm².

The step of subculturing the cells after the cells spread out is specifically as follows: when the cells of the primary culture reach a confluence of 90%-100%, removing the medium, washing the cells with PBS, performing digestion in a 37° C. incubator for 7-10 minutes using a TrypLE Express solution, terminating the digestion with an RPE cell medium, gently blowing the cells off with a pipette, centrifuging to remove the digestive reagent, resuspending the cells in an RPE cell medium, seeding the cells into a cell culture container precoated with extracellular matrix to allow subculture; when the cells reach a confluence of 90%-100%, repeating the above steps for repetitive subcultures; the step of seeding for subculture is performed at a cell density greater than or equal to 2×10⁴ cells/cm². The human RPE cells obtained by the present invention can be subcultured for at least 5 passages.

The extracellular matrix is preferably Matrigel or Gelatin.

The cell culture container is preferably a culture plate, a culture dish or a culture flask.

Formula of the cell medium used in the primary culture and the subculture is as follows: 100 mL of the cell medium comprises 10 mL of fetal bovine serum, 2 mL of 50× B-27, 1 mL of 100× penicillin-streptomycin solution, 1 mL of 100× non-essential amino acids solution, 1 mL of 100× glutamine, 0.1 mL of 1000× taurine, and the balance is a DMEM/F12 (3:1) mixed medium; the DMEM/F12 (3:1) mixed medium is prepared by blending a DMEM/F12 (1:1) medium and a DMEM medium in a volume ratio of 3:2.

Cryopreservation of the human RPE cells is specifically as follows: the RPE cells primarily cultured and subcultured can be cryopreserved using known methods. The cryoprotectant is a cell culture medium containing 10% by volume of DMSO. The cryopreserved cells can be recovered and exhibit the same characteristics.

The pluripotent stem cell-derived RPE cells prepared by the method of the invention show characteristics closely similar to those of the human fetal RPE cells, that they have typical RPE morphology, express specific molecular markers PAX6, OTX2, ZO-1 and RPE65, and exhibit normal physiological functions such as polarized secretion of cell factor PEDF. Therefore, the method can provide seed cells for related research and treatment.

Compared with prior art, the present invention has the following advantages.

1. Widely applicable. The invention can be applied in preparation, purification, and expansion of human RPE cells, including RPE cells derived from human pluripotent stem cell lines (hESC line and hiPSC line) by the two-dimensional protocol or the three-dimensional induced differentiation protocol.

2. Simple and convenient. The preparation, purification and expansion of RPE cells are performed mainly by mechanical separation and enzymatic digestion, without the aid of complicated instruments and techniques such as flow cytometry, magnetic bead separation and reporter gene labeling. The digestive reagents, Dispase II and TrypLE Express as used for digesting the RPE cells, are mild, with little damage to the RPE cells. By using the methods of the present invention, the inventors have succeeded in preparing RPE cells derived from three different hPSC lines, with at least three replicates for each cell line. The whole set of experimental techniques is simple and easy to learn, and can be quickly mastered by beginners, with low cost and good efficiency.

3. The hPSC-derived human RPE cells obtained by the method of the present invention show good capacity in expansion and subculture, good growth characteristics and high yield, allowing mass production which can reduce batch-to-batch differences. When seeded on a Matrigel-coated culture plate at a density greater than 2×10⁴ cells/cm², the cells are able to reach a confluence of more than 90% after about 7 days of growth. The RPE cells prepared by the method of the invention can be expanded to achieve a nearly 15-fold expansion, allowing a 3000-fold expansion in three successive subcultures. The RPE cells obtained by the method of the invention have a doubling time of about 1.52 days such that it takes only 7 days for the cells to expand in every passage, can be subcultured for at least 5 passages, and can be recovered from cryopreservation.

4. The hPSC-derived human RPE cells obtained by the method of the present invention exhibit typical RPE morphology and pigmentation, similar to those of the human fetal RPE cells.

5. The RPE cells obtained by the method of the present invention express RPE specific molecular markers PAX6, OTX2, ZO-1 and RPE65, similar to those of the human fetal RPE cells.

6. The RPE cells obtained by the method of the present invention also exhibit functional characteristics similar to those of RPE cells in vivo, including transepithelial electrical resistance and polarized secretion of cytokines such as PEDF, suggesting good application prospects.

7. The RPE cells obtained by the method of the present invention have a high purity of up to 98%, and can be widely applied to related fields as research materials and thereby the bottleneck of seed cells is solved.

In view of the above, the present invention has established a novel technique for preparing human RPE cells from hPSCs, allowing mass production of human RPE cells. The obtained human RPE cells are closely similar to human fetal RPE cells in growth characteristics, morphology, expressions of specific molecules and functions, and have high purity, suggesting that the RPE cells prepared by the method of the present invention have broad application prospects in related fields such as tissue engineering, regenerative medicine, disease mechanism and drug screening, and in particular, allows provision of seed cells for research and treatment of retinal diseases so as to solve the bottleneck of limited sources of human retinal pigment epithelial cells and lack of RPE transplant donors. The methods of the present invention reach the world leading level in the field, and have great significance for the restoration of visual function of retinal diseases patients.

### BRIEF DESCRIPTION OF THE DRAWINGS

FIG. 1 is an inverted microscope image of hPSCs.

FIG. 2 is an inverted microscope image (40×) of RPE cells formed after 26 days of induced differentiation of hPSCs.

FIG. 3 shows inverted microscope images (40×) of 3D-RPE spheroids formed by induced differentiation of hPSCs in suspension. The 3D-RPE spheroids are adherent to (A) one side of neural retina (NR), or (B) one side of a cell cluster.

FIG. 4 is an image (100×) of single RPE cells which were obtained by mechanical separating, purifying and enzymatically digesting the 3D-RPE spheroids.

FIG. 5 is an image (200×) taken after a seven-day primary culture of the RPE cells prepared in embodiment 6.

FIG. 6 is an image (100×) taken after a seven-day culture of the fifth passage of the RPE cells prepared in embodiment 6.

FIG. 7 shows the result of immunofluorescence.

FIG. 8 shows the result of transepithelial electrical resistance (TEER) test.

FIG. 9 shows the result of PEDF measurement by ELISA.

FIG. 10 shows the result of flow cytometry.

### DETAILED DESCRIPTION OF THE EMBODIMENTS

The following embodiments are for further describing this invention rather than limiting the invention.

### Embodiment 1: Expansion of Human Pluripotent Stem Cells

Three human pluripotent stem cell (hPSC) lines were used in the study. BC1-GFP-hiPSC line and BC1-hiPSC line were obtained from friends while a third hiPSC line was purchased from Life Technologies Corporation (Gibco® Episomal hiPSC Line, A18945). The cells were seeded onto 6-well plates coated with extracellular matrix MatriGel (Corning, 354277) and expanded in mTeSR1 media. When the cells reached a confluence of 80%-90%, they were digested with 0.5 mM EDTA (Life, 15575-038), and passaged in a ratio of 1:8 to 1:12. The inverted microscope image showed that the cells were sheet-shaped and exhibited colony-like growth wherein the cells in the colony were compactly arranged with unclear boundaries (FIG. 1). Accordingly, human pluripotent stem cells (hPSCs) were obtained by expansion.

### Embodiment 2: Induced Differentiation of hPSCs Towards Retinal Cells Including Pigment Epithelium Cells

Induced differentiation of hPSCs towards retinal cells including pigment epithelium cells was conducted according to a reported protocol (Xiufeng Zhong, et al. Generation of three-dimensional retinal tissue with functional photoreceptors from human iPSCs. Nat Commun. 2014 Jun. 10; 5: 4047), and the day of initiation of the differentiation (when the expansion medium of the hPSCs was replaced with a differentiation medium, or when embryoid bodies were prepared) was regarded as day 0 of the differentiation.

### Embodiment 3: Preparing and Culturing hPSC-Derived 3D-RPE Spheroids

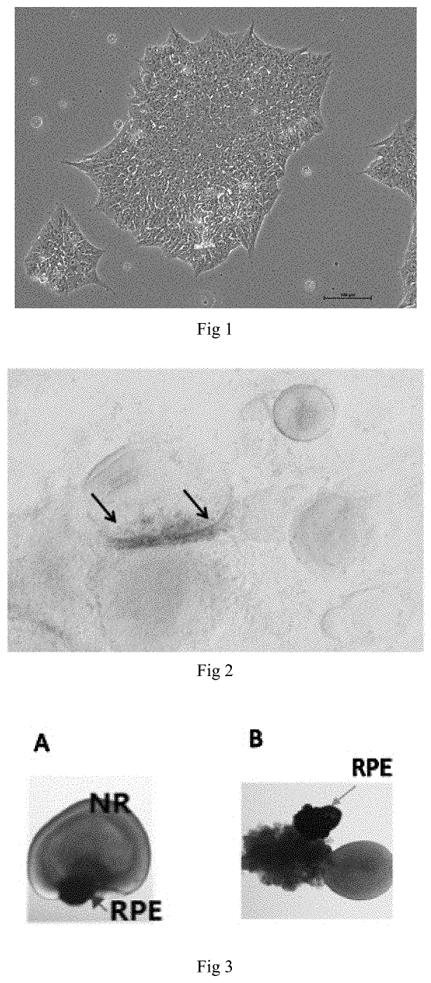

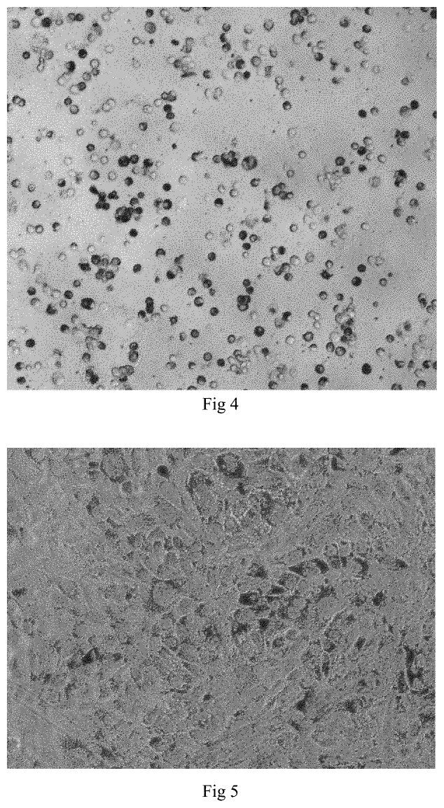

The retinal cells including pigment epithelium cells which were obtained at the fourth week of the induced differentiation of hPSCs were observed under microscope. Neural retina (NR) and RPE, which slightly rose up, shaped like a ring, and had high refractivity, could both be identified. RPE could grow around the NR, or grow individually in the form of dots or sheets (FIG. 2). The NR and its surrounding RPE were scraped using a self-made tungsten needle or a 1 mL syringe, and then transferred to a low attachment culture dish to allow suspension culture in an RPE cell medium. The formula of the cell medium was as follows: 100 mL of the cell medium comprises 10 mL of fetal bovine serum (Gibco, 10099-141), 2 mL of 50× B-27 supplement (Gibco, 12587-010), 1 mL of 100× penicillin-streptomycin solution (Gibco, 15240), 1 mL of 100× non-essential amino acids solution (Gibco, 11140-050), 1 mL of 100× glutamine (Gibco, 35050-061), 0.1 mL of 1000× taurine (Sigma, T-0652), and the balance is a DMEM/F12 (3:1) mixed medium; the DMEM/F12 (3:1) mixed medium is prepared by blending a DMEM/F12 (1:1) medium (Gibco, C11330500BT) and a DMEM medium (Gibco, C11995500BT) in a volume ratio of 3:2. The rest of the cells, at any time from the fourth week to the sixth week, could be scraped by a cell scraper and transferred to low attachment culture dishes to allow suspension culture in a conventional incubator using a RPE cell medium. The suspension-cultured RPE usually curled into spheroids (i.e. the 3D-RPE spheroids), which could be adherent to one side of NR or another cell cluster (FIG. 3), or could be free-floating.

### Embodiment 4: Collecting, Mechanically Separating and Purifying the hPSC-Derived 3D-RPE Spheroids

After six weeks of differentiation of hPSCs, all suspension-cultured 3D-RPE spheroids (including free-floating spheroids and those adherent to one side of NR or another cell cluster) were collected and placed in 60 mm culture dishes. Under a dissecting microscope, the NR and the non-RPE clusters were removed from the RPE spheroids, and the pigmented 3D-RPE spheroids were retained.

### Embodiment 5: Separating and Purifying the hPSC-Derived Human RPE Sheets

The 3D-RPE spheroids purified in embodiment 4 were digested at 37° C. for 8-15 minutes in a Dispase II solution (Sigma, D4693-1G) having a mass fraction of 1%, and then washed with 1×PBS for three times after the digestive reagent was removed. On the surface of 3D-RPE spheroids were RPE which were black while inside were non-RPE cell clusters. Under a dissecting microscope, the RPE were separated from the non-RPE cell clusters using a tungsten needle and then RPE cell sheets were collected.

### Embodiment 6: Preparing a Single Cell Suspension of hPSC-Derived Human RPE Cells

The purified RPE cell sheets were digested in a TrypLE Express solution (Gibco, 12604-013) at 37° C. for 7-10 minutes, and then a same volume of RPE cell medium was added into the solution to terminate the digestion. The cells were then dispersed by blowing with a 1 mL pipette tip until there was no visible cell cluster, filtered through a 70 μm strainer, and centrifuged at 1000 rpm at room temperature. The supernatants were removed and the cell pellets were retained. The cells were then resuspended in a RPE cell medium (identical to the medium in embodiment 3) to obtain a single cell suspension, and thereby hPSC-derived RPE cells were obtained. Through the above protocols, RPE cells were obtained from all the three hPSC lines in embodiment 1.

### Embodiment 7: Primary Culture of hPSC-Derived Human RPE Cells

The total number of cells in a single cell suspension of hPSC-derived human RPE cells, which is obtained by the method of the present invention, was counted on a hemocytometer. The cells were seeded onto 6-well plates precoated with Matrigel at a cell density of 5×10⁴ cells/cm², and primary culture was performed at 37° C. under 5% CO₂ and saturated humidity with an RPE cell medium. Thirty minutes after the cells were seeded, they began to attach to the plate. The cells were circular, bright, and showed high refractivity, and most of the cells carried pigment particles (FIG. 4). At the seventh day of the primary culture, the RPE cells prepared by the method reached a confluence; they were polygonal in shape and filled with pigment (FIG. 5). Primary cultures of the PRE cells derived from the three hPSC lines showed the similar results.

### Embodiment 8: Subculture of hPSC-Derived Human RPE Cells

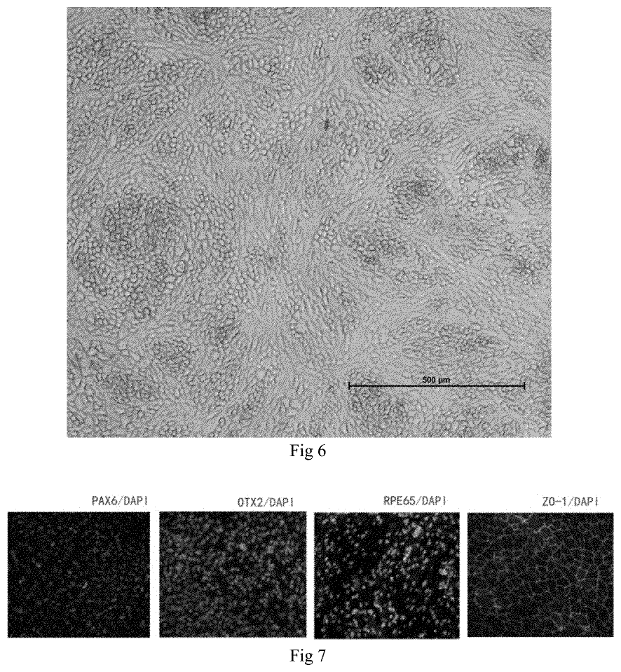

Primary culture of RPE cells derived from hiPSCs (Gibco® Episomal hiPSC Line, purchased from Life Technologies Corporation) was performed for 7-8 days to reach a 100% confluence when they could be passaged. The PRE cells to be passaged were collected, and washed with PBS twice after the medium was removed. The RPE cells were then digested in a TrypLE™ Express solution at 37° C. for 7-10 minutes, and then an RPE cell medium (identical to that in embodiment 3) was added to neutralize the digestive reagent. The digested PRE cells were collected and counted. The digestive solution was removed by centrifugation, and the cells were resuspended in an RPE cell medium. The cells were then seeded onto culture plates coated with Matrigel at a cell density of 2-5×10⁴ cells/cm², and subculture was performed at 37° C. under 5% CO₂ in an incubator. The RPE cells prepared by this method had high purity and proliferative capacity such that they could be subcultured once every week for at least five passages; the subcultured cells retained PRE cell morphology that they were polygonal in shape, arranged like pebbles and slightly pigmented (FIG. 6). The pigment particles in the cells gradually decreased as the number of passages increased, which is similar to the culturing characteristics of human fetal RPE cells. In addition, RPE cells derived from the BC1-GFP-hiPSC line and the BC1-hiPSC line, which were subcultured and expanded by the above protocols, also presented the same results.

### Embodiment 9: Cryopreservation and Recovery of hPSC-Derived Human RPE Cells

After RPE cells derived from hiPSCs (Gibco® Episomal hiPSC Line, purchased from Life Technologies Corporation) grew to confluence, they were digested by the protocol in embodiment 8, centrifuged and then subjected to conventional cell cryopreservation. The cryoprotectant was an RPE cell medium supplied with 10% by volume of DMSO (the RPE cell medium was same as used in embodiment 3). The cryopreserved cells could be recovered and cultured under the same conditions of the subculture process. The recovered cells had high proliferative capacity such that they could reach a confluence of 100% in 7-8 days and exhibit the similar typical characteristics of RPE cells. In addition, RPE cells derived from the BC1-GFP-hiPSC line and the BC1-hiPSC line, which were cryopreserved and recovered by the above protocols, also presented the same results.

### Embodiment 10: Growth Dynamics Analysis of hPSC-Derived Human RPE Cells

Growth potential of RPE cells: Passage 5 of the cells obtained by induced differentiation of BC1-GFP-hiPSC were seeded onto culture plates coated with Matrigel at a density of 5×10⁴ cells/cm² at 37° C. under 5% CO₂ and saturated humidity with an RPE cell medium, and subcultured every seven days. Successive subcultures were performed until passage 10, and the total number of cells obtained in every passage was counted on a hemocytometer. The BC1-GFP-RPE cells (i.e. the RPE cells obtained by induced differentiation of BC1-GFP-hiPSC) exhibited stable expansion, and could be subcultured for more than 5 passages. When seeded on a Matrigel-coated culture plate at a density greater than 2×10⁴ cells/cm², the cells were able to reach a confluence of more than 90% after about 7 days of growth. The RPE cells prepared by the method of the invention can be expanded to achieve a nearly 15-fold expansion, allowing a 3000-fold expansion in three successive subcultures.

Growth curve of RPE cells: Passage 5 of the cells obtained by induced differentiation of BC1-GFP-hiPSC were seeded onto a 96-well plate with an RPE cell medium. Three wells of cells were collected, digested and counted every day for seven successive days. A growth curve was plotted with cell number as ordinate against days as abscissa. The RPE cells obtained by the method of the invention have a doubling time of about 1.52 days such that it takes only 7 days for the cells to expand in every passage.

### Embodiment 11: Immunofluorescence Identification of Specific Molecular Markers Expression of hPSC-Derived Human RPE Cells

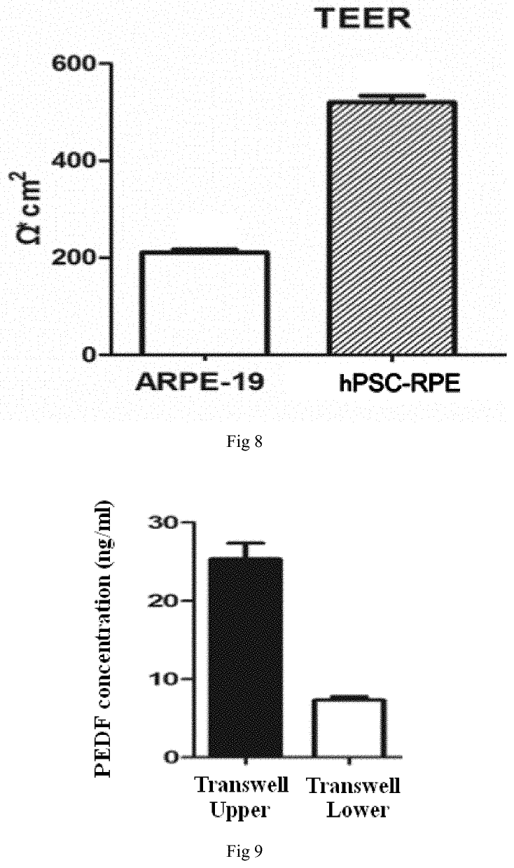

A portion of RPE cells prepared by the present invention (the RPE cells obtained by induced differentiation of BC1-GFP-hiPSC), when subcultured, were seeded onto coverslips coated with Matrigel and cultured with an RPE cell medium containing 10% of FBS. When the cells reached a confluence of 100%, the FBS was removed, and the culture proceeded in a serum-free RPE cell culture solution. Formula of the serum-free RPE cell medium is as follows: 100 mL of the cell medium comprises 2 mL of 50× B-27 supplement (Gibco, 12587-010), 1 mL of 100× penicillin-streptomycin solution (Gibco, 15240), 1 mL of 100× non-essential amino acids solution (Gibco, 11140-050), 1 mL of 100× glutamine (Gibco, 35050-061), 0.1 mL of 1000× taurine (Sigma, T-0652), and the balance is a DMEM/F12 (3:1) mixed medium; the DMEM/F12 (3:1) mixed medium is prepared by blending a DMEM/F12 (1:1, Gibco, C11330500BT) medium and a DMEM medium (Gibco, C11995500BT) in a volume ratio of 3:2. At different time intervals after the culture began, the coverslips were taken out, washed with PBS once, and placed on ice to allow cell fixation using a 4% formaldehyde solution for 5-10 minutes. Then the coverslips were washed with PBS for three times, and added into a blocking solution (PBS containing 10% of normal donkey serum and 0.25% of Triton X-100) to allow blocking at room temperature for 1 hour. The samples were then incubated with primary antibodies at 4° C. overnight. The primary antibodies were: PAX6 (mouse, 1:50, DSHB), OTX2 (rabbit, 1:200, abcam), RPE65 (mouse, 1:500, abcam), ZO-1 (mouse, 1:400, Life Technologies), and CHX10 (sheep, 1:200, Millipore). The next day, the cells were washed with PBS for three times and then incubated with fluorescence-labeled secondary antibodies (1:500, Life Technologies) at room temperature for 1 hour. After incubated with secondary antibodies, the cells were washed with PBS and staining was performed with DAPI for 10 minutes. After washing with PBS for three times, observation and photographing were performed with an Olympus fluorescence microscope. The RPE cells, which were prepared by the method of the invention, expressed RPE cell specific molecular markers PAX6, OTX2, RPE65 and ZO-1 (FIG. 7), but did not express retinal neural precursor cell marker CHX10. The RPE cells which were obtained from induced differentiation of the hiPSC line (Gibco® Episomal hiPSC Line, A18945, purchased from Life Technologies) and the BC1-hiPSC line also presented the same results.

### Embodiment 12: Function Analysis of the RPE Cells Prepared by the Method of the Present Invention

Transepithelial electrical resistance test: A portion of the RPE cells prepared by the present invention (the RPE cells obtained by induced differentiation of BC1-GFP-hiPSC), when subcultured, were seeded onto Transwell (Corning, 0.4 μm transparent polyester membrane, product number: 3470) precoated with Matrigel, and cultured using an RPE cell medium containing 10% of FBS (identical to the RPE cell medium in embodiment 3). When the cells reached a confluence of 100% (about 7-8 days later), the FBS was removed, and the culture proceeded in a serum-free RPE cell medium (identical to the medium in embodiment 11). 6-8 weeks after the culture began, a transepithelial electrical resistance (TER) measurement was carried out with an epithelial voltohmmeter (WPI, EVOM2). The electrodes were sterilized by soaking in 75% alcohol and rinsed with Hank's balanced salt solution before the measurement. The TER value of the Transwell coated with Matrigel was measured as the background value. The actual TER value was calculated by subtracting the background value from the display value. The TER measurement of each well was performed at three spots to obtain an average value, and three replicates were performed. An RPE cell line ARPE-19 was taken as a control group. The RPE cells (hPSC-RPE) prepared by the invention exhibited high electrical impedance (520.3±23.6 Ω*cm²), significantly higher than that of ARPE-19 cells (210.7±10.5 Ω*cm²) (FIG. 8), suggesting that the RPE cells prepared by the present invention exhibit an electrical impedance function similar to RPE cells in vivo and have high electrical impedance better than that of ARPE-19 cell line. The RPE cells which were obtained from induced differentiation of the hiPSC line (Gibco® Episomal hiPSC Line, A18945, purchased from Life Technologies) and the BC1-hiPSC line also presented the same results.

Measurement of PEDF Secretion:

The RPE cells prepared by the present invention (the RPE cells obtained by induced differentiation of BC1-GFP-hiPSC) were seeded onto Transwell at a density of 5×10⁴ cells/cm² and cultured under a condition identical to that in the transepithelial electrical resistance test section. When the TER value was determined to be greater than 200 Ω/cm², the cells were washed with PBS for three times, and then the medium was replaced with a serum-free RPE cell culture solution (identical to that in embodiment 11), with 120 μL in upper compartment and 1 mL in the lower compartment. The cells were cultured in an incubator at 37° C. under 5% CO₂ and saturated humidity for 24 hours, and then the media in the upper and lower compartments were collected respectively. Human pigment epithelium-derived factor (PEDF) levels in the culture solutions in the upper and lower compartments were measured by ELISA. The ELISA kit was purchased from Cusabio Technology LLC (product number: CSB-EO8818h). The measurement was conducted according to the manufacturer's specification. The RPE cells prepared by the present invention exhibited polarized secretion of PEDF wherein the PEDF level in the Transwell upper compartment (25.3±3.5 ng/mL) was higher than that in the lower compartment (7.3±0.8 ng/mL) (FIG. 9), which was similar to that of human RPE cells in vivo. The RPE cells which were obtained from induced differentiation of the hiPSC line (Gibco® Episomal hiPSC Line, A18945, purchased from Life Technologies) and the BC1-hiPSC line also presented the similar results.

### Embodiment 13: Flow Cytometry Analysis on Purity of the RPE Cells Prepared by the Present Invention

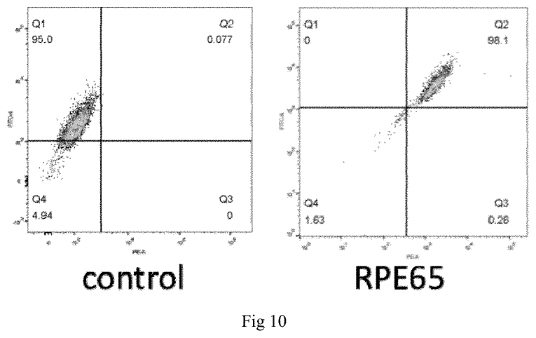

After the RPE cells prepared by the present invention (the RPE cells obtained by induced differentiation of BC1-GFP-hiPSC) was subcultured for 7-8 days to reach a confluence of 100%, the serum-containing RPE cell culture solution was replaced with a serum-free RPE cell culture solution (identical to that in embodiment 11) and then the culture proceeded for 6-8 weeks. The attached RPE cells were digested with a TrypLE™ Express solution to give a single cell suspension. The suspension was then centrifuged at 1000 rpm for 5 minutes, then the pellet was resuspended in 2 ml of 1% formaldehyde solution for cell fixation for 15 minutes. Then the suspension was centrifuged at 1000 rpm for 5 minutes and the cells were washed with a PBS solution containing 0.04% of triton-X-100 and 2% of donkey serum. This step was repeated twice. Primary antibody RPE65 (mouse, abcam, cat. AB78036) was diluted using a PBS solution containing 0.25% of triton-X-100 and 2% of donkey serum, then the cells were incubated with the primary antibody for 1 hour wherein the concentration of the primary antibody was 2 μg/1×10⁶ cells. The cells were washed according the aforementioned method and then incubated with Alexa 555-labeled donkey anti-mouse secondary antibody (1:500, Life Technologies) at room temperature for 30 minutes. After washing, the cells were resuspended in 500 μL PBS for analysis. The cells without primary antibody were taken as parallel negative control. The flow cytometry was purchased from BD company (model: LSRFortessa). The RPE cells prepared by the present invention expressed specific molecular marker RPE65, and positive cells accounted for 98.1% (FIG. 10), indicating that the RPE cells prepared by the present invention have a high purity and thus can be applied to related researches. The RPE cells which were obtained from induced differentiation of the hiPSC line (Gibco® Episomal hiPSC Line, A18945, purchased from Life Technologies) and the BC1-hiPSC line also presented the same results.

What is claimed is:

1. A preparation method for human pluripotent stem cell-derived human retinal pigment epithelial cells, comprising following steps:
collecting 3D-PRE spheroids derived from human pluripotent stem cells,
performing mechanical separation to remove non-pigmented non-RPE cells and clusters and retain a pigmented RPE cell sheet,
enzymatically digesting the pigmented RPE cell sheet to obtain an RPE single cell suspension, and thereby obtaining the human pluripotent stem cell-derived human retinal pigment epithelial cells.

2. The preparation method according to claim 1, wherein, the 3D-PRE spheroids are prepared by inducing directed differentiation of the human pluripotent stem cells into retinal cells including RPE cells, collecting adherent cells including the RPE cells by scraping, and performing suspension culture to obtain the 3D-RPE spheroids; the 3D-RPE spheroids are free-floating, adherent to one side of a neural retinal cup, or adherent to one side of another cell cluster.

3. The preparation method according to claim 1, wherein, the step of performing mechanical separation to remove the non-pigmented non-RPE cells and clusters and retain a pigmented RPE cell sheet, comprises: transferring all the 3D-RPE spheroids into a cell culture container, digesting in a 37° C. water bath for 8-15 minutes using a digestive reagent, washing with 1×PBS for a plurality of times after the digestive reagent was aspirated, separating the pigmented RPE cell sheet from the non-pigmented non-RPE cells and clusters using a tungsten needle, and retaining the pigmented RPE cell sheet.

4. The preparation method according to claim 1, wherein, the step of enzymatically digesting the pigmented RPE cell sheet to obtain the RPE single cell suspension, comprises: digesting the pigmented RPE cell sheet with a TrypLE Express solution in a 37° C. water bath for 7-10 minutes, terminating digestion, filtering the cells through a 70-100 μm strainer, centrifuging to remove the digestive reagent, and resuspending in an RPE cell medium to obtain the RPE single cell suspension.

5. An expansion method for human pluripotent stem cell-derived human retinal pigment epithelial cells, comprising:
centrifuging the RPE single cell suspension of claim 1 and removing a supernatant;
resuspending in an RPE cell medium and seeding into a cell culture container precoated with extracellular matrix to allow primary culture;
after cells spread out, subculturing the cells to obtain the human pluripotent stem cell-derived human retinal pigment epithelial cells.

6. The expansion method according to claim 5, wherein, the step of seeding for primary culture is performed at a cell density greater than or equal to 5×10⁴ cells/cm².

7. The expansion method according to claim 5, wherein, the step of subculturing the cells after the cells spread out comprises:
when the cells of the primary culture reach a confluence of 90%-100%, removing the medium, washing the cells with PBS, performing digestion in a 37° C. incubator for 7-10 minutes using a TrypLE Express solution, terminating the digestion with an RPE cell medium, gently blowing the cells off with a pipette, centrifuging to remove the digestive reagent, resuspending the cells in an RPE cell medium, seeding the cells into a cell culture container precoated with extracellular matrix to allow subculture;
when the cells reach a confluence of 90%-100%, repeating the above steps to allow repetitive subcultures;
wherein, the step of seeding for subculture is performed at a cell density greater than or equal to 2×10⁴ cells/cm².

8. The expansion method according to claim 5, wherein the extracellular matrix is Matrigel or Gelatin.

9. The expansion method according to claim 5, wherein, a formula of the cell medium used in the primary culture and the subculture is as follows: 100 mL of the cell medium comprises 10 mL of fetal bovine serum, 2 mL of 50× B-27 supplement, 1 mL of 100× penicillin-streptomycin solution, 1 mL of 100× non-essential amino acids solution, 1 mL of 100× glutamine, 0.1 mL of 1000× taurine, and the balance is a DMEM/F12 mixed medium; the DMEM/F12 mixed medium is prepared by blending a DMEM/F12 (1:1) medium and a DMEM medium in a volume ratio of 3:2.
